(19) United States
(12) Patent Application Publication
(10) Pub. No.: US 2020/0405591 A1
(43) Pub. Date: Dec. 31, 2020

(54) SPRAYABLE COMPOSITION

(71) Applicant: L'OREAL, Paris (FR)

(72) Inventors: Tomoko MIZUNO, Kanagawa (JP); Kazuhiko MARUYAMA, Kanagawa (JP)

(21) Appl. No.: 16/976,231

(22) PCT Filed: Feb. 26, 2019
(86) PCT No.: PCT/JP2019/008257

§ 371 Date: Aug. 27, 2020

(30) Foreign Application Priority Data
Mar. 1, 2018 (JP) 2018-036344

(51) Int. Cl. A61K 8/04; A61Q 19/00; A61K 8/73; A61K 8/37; A61K 8/92; A61K 8/60

(52) U.S. Cl. CPC A61K 8/046; A61Q 19/007; A61K 8/731; A61K 8/604; A61K 8/737; A61K 8/922; A61K 8/375

(57) ABSTRACT

The present invention relates to a composition comprising (a) at least one microcrystalline cellulose; (b) at least one nonionic surfactant of ester type; (c) at least one surfactant of alkylpolyglycoside type; (d) at least one polysaccharide hydrophilic gelling agent other than the ingredient (a); (e) at least one oil; and (f) water. The composition according to the present invention has good sprayability and stability, and can provide a smooth skin finish.

### TECHNICAL FIELD

The present invention relates to a sprayable composition which is preferably in the form of an O/W emulsion.

### BACKGROUND ART

In recent years, spraying products have been used in the field of cosmetic skincare products.

In general, compared with conventional cosmetic liquid products (direct application by hand), spraying products can provide a more refreshing sense of use after the mist is blown.

However, there are the following problems in case of using a spray container for spraying an emulsion (e.g., a milky lotion) composition.

First, a highly viscous emulsion cannot blow out in a fine mist. Second, in the case that the viscosity of a highly viscous emulsion is decreased for spraying in order to realize a fine mist, the stability of the emulsion deteriorates such that separation, creaming, and the like are observed. Third, an emulsion usually contains a certain degree of oils and surfactants which often cause a sticky feeling, and therefore, the refreshing sense during use from the beginning to the end by using mist products is damaged due to this sticky feeling.

Thus, there is a need for a sprayable composition with a good sprayability (fine mist with refreshing feeling after being sprayed out) on use (especially during application), a good stability (no separation), together with no sticky feeling after application (smooth skin finish).

### DISCLOSURE OF INVENTION

An objective of the present invention is to provide a composition which has good sprayability and stability, and can provide a smooth skin finish.

The above objective can be achieved by a composition comprising:

(a) at least one microcrystalline cellulose;

(b) at least one nonionic surfactant of ester type;

(c) at least one surfactant of alkylpolyglycoside type;

(d) at least one polysaccharide hydrophilic gelling agent other than the ingredient (a);

(e) at least one oil; and

(f) water.

The (a) microcrystalline cellulose may be in the form of a particle, and the particle preferably has an average particle size of 50 μm or less, preferably 10 μm or less, and more preferably less than 1 μm.

In another embodiment, the (a) microcrystalline cellulose may be in the form of a fiber, and the fiber preferably has an average fiber diameter of 2-5 nm.

The amount of the (a) microcrystalline cellulose in the composition may be from 0.05% to 5% by weight, preferably from 0.08% to 3% by weight, and more preferably from 0.1% to 2% by weight, relative to the total weight of the composition.

The (b) nonionic surfactant of ester type may be selected from the group consisting of monounsaturated esters, polyglyceryl diesters, and mixtures thereof

The (b) nonionic surfactant of ester type may be a mixture comprising:

i) at least one monounsaturated ester of formula (A)

R¹—C(O)—O—R² (A)

wherein

R¹ and R² represent, respectively, a C₁₈ to C₄₄ fatty chain, at least one of R¹ or R² is monounsaturated;

ii) at least one polyglyceryl diester of formula (B)

R³—C(O)—(O—CH₂—CH(OH)—CH₂)ₙ—O—C(O)—R⁴ (B)

wherein

R³ and R⁴ represent, respectively, a saturated C₁₈ to C₄₄ fatty chain, linear or branched, and n is an integer between 2 and 6; and

iii) at least one C₁₀-C₃₀ fatty alcohol,

preferably, the (b) nonionic surfactant of ester type is a mixture comprising at least one monounsaturated ester of formula (A) wherein R¹ and R² represent, respectively, a C₁₈-C₃o fatty chain, and at least one of R¹ or R² is monounsaturated; at least one polyglyceryl diester of formula (B) wherein, R³ and R⁴ each represent a saturated C₂₀ to C₃₄ fatty chain, linear or branched; and cetyl alcohol.

The (b) nonionic surfactant of ester type may further comprise a diester of a C₁₄-C₂₂ fatty acid with a polyglycerol and/or a fatty alcohol containing from 10 to 30 carbon atoms.

The (b) nonionic surfactant of ester type may be a mixture of polyglyceryl-6 distearate, jojoba esters, polyglyceryl-3 beeswax, and cetyl alcohol.

The amount of the (b) nonionic surfactant of ester type in the composition may be from 0.05% to 10% by weight, preferably from 0.1% to 5% by weight, and more preferably from 0.5% to 3% by weight, relative to the total weight of the composition.

The (c) surfactant of alkylpolyglycoside type may be selected from the compound of formula (I):

R(O)(G)ₓ (I)

in which the radical R is a linear or branched C₁₂-C₂₂ alkyl radical, x ranges from 1 to 5 and G is a saccharide residue chosen from the group of glucose, dextrose, saccharose, fructose, galactose, maltose, maltotriose, lactose, cellobiose, mannose, ribose, dextran, talose, allose, xylose, levoglucan, cellulose and starch; preferably, G is glucose.

The amount of the (c) surfactant of alkylpolyglycoside type in the composition may be from 0.01% to 5% by weight, preferably from 0.03% to 1% by weight, and more preferably from 0.05% to 0.5% by weight, relative to the total weight of the composition.

The (d) polysaccharide hydrophilic gelling agent may be selected from xanthan gum, carob gum and mixtures thereof.

The amount of the (d) polysaccharide hydrophilic gelling agent in the composition may be from 0.0005% to 0.5% by weight, preferably from 0.0008% to 0.2% by weight, and more preferably from 0.001% to 0.1% by weight, relative to the total weight of the composition.

The amount of the (e) oil in the composition may be from 0.1% to 20% by weight, preferably from 0.5% to 10% by weight, and more preferably from 1% to 8% by weight, relative to the total weight of the composition.

The amount of the (f) water in the composition may be from 50% to 95% by weight, preferably from 55% to 90% by weight, and more preferably from 60% to 85% by weight, relative to the total weight of the composition.

The composition according to the present invention may further comprise (g) at least one anionic surfactant.

The amount of the (g) anionic surfactant(s) in the composition may be from 0.01% to 1% by weight, preferably from 0.05% to 0.5% by weight, and more preferably from 0.08% to 0.2% by weight, relative to the total weight of the composition.

The composition according to the present invention may be a cosmetic composition, preferably a skin cosmetic composition, and more preferably a skincare cosmetic composition.

The present invention also relates to a cosmetic process for a keratin substance, preferably skin, comprising applying to the keratin substance the composition according to the present invention.

### BEST MODE FOR CARRYING OUT THE INVENTION

After diligent research, the inventors have discovered that it is possible to provide a composition which has good sprayability and stability, and can provide a smooth skin finish.

Thus, one of the aspects of the present invention relates to a composition comprising:

(a) at least one microcrystalline cellulose;

(b) at least one nonionic surfactant of ester type;

(c) at least one surfactant of alkylpolyglycoside type;

(d) at least one polysaccharide hydrophilic gelling agent other than the ingredient (a);

(e) at least one oil; and

(f) water.

The composition according to the present invention is sprayable, and can blow a fine mist when being sprayed out.

The composition according to the present invention can provide a refreshing feeling after application, in particular a refreshing feeling immediately after application. Therefore, a fresh sensation can start from the beginning of use and continue during use.

The composition according to the present invention can provide no sticky feeling after application, and therefore, can provide a smooth skin finish.

The composition according to the present invention has good stability such that it can be homogeneous and cause no separation of ingredients in the composition, e.g., phase separation of oil and aqueous phases.

Hereafter, the composition according to the present invention will be described in a detailed manner.

[Composition]

(Microcrystalline Cellulose)

The composition according to the present invention comprises (a) at least one microcrystalline cellulose. If two or more microcrystalline celluloses are used, they may be the same or different.

There is no limitation about the (a) microcrystalline cellulose to be used in the composition according to the present invention.

The (a) microcrystalline cellulose may be in the form of a particle, and the particle preferably has an average particle size of 50 μm or less, preferably 10 μm or less, and more preferably less than 1 μm. The average particle size means volume average particle size. As the (a) microcrystalline cellulose in the form of a particle, Avicel® PC 611 (FMC Corporation) may be used.

In another embodiment, the (a) microcrystalline cellulose may be in the form of a fiber, and the fiber preferably has an average fiber diameter of 2-5 nm. The average fiber diameter means volume average fiber diameter. As the (a) microcrystalline cellulose in the form of fiber, RHEOCRYSTA® C-2SP (DKS Co. Ltd.) may be used.

It may be preferable that the (a) microcrystalline cellulose be the isolated, crystalline portion of cellulose fibers from wood pulp which can be used in colloidal (i.e. co-processed with a soluble hydrocolloid) or non-colloidal form.

In one embodiment, the (a) microcrystalline cellulose may be used in colloidal form having a particle size of <1 um, i.e., a rod-like particle shape. The initial viscosity is preferably selected in the range of 30 to 150 cps as a 1 to 3 wt % dispersion. Even more preferably, the (a) microcrystalline cellulose additionally has a sieve fraction of +60 mesh of not more than 0.1% by weight and +325 mesh of not more than 75% by weight.

The (a) microcrystalline cellulose may be used alone or in the form of a mixture with sodium carboxymethylcellulose as the coprocessd microcrystalline cellulose.

It may be more preferable that the (a) microcrystalline cellulose is that included in the product Avicel® PC 611 (FMC Corporation) comprising microcrystalline cellulose and sodium carboxymethylcellulose (CELLULOSE GUM) in a ratio of 85/15 by weight, or RHEOCRYSTA® C-2SP (DKS Co. Ltd.) (aqueous dispersion of 2% by weight of microcrystalline cellulose).

The amount of the (a) microcrystalline cellulose in the composition according to the present invention may be 0.05% by weight or more, preferably 0.08% by weight or more, and more preferably 0.1% by weight or more, relative to the total weight of the composition.

The amount of the (a) microcrystalline cellulose in the composition according to the present invention may be 5% by weight or less, preferably 3% by weight or less and more preferably 2% by weight or less, relative to the total weight of the composition.

The amount of the (a) microcrystalline cellulose in the composition according to the present invention may be from 0.05% to 5% by weight, preferably from 0.08% to 3% by weight, and more preferably from 0.1% to 2% by weight, relative to the total weight of the composition.

(Ester Type Nonionic Surfactant)

The composition according to the present invention comprises (b) at least one nonionic surfactant of ester type. If two or more nonionic surfactants of ester type are used, they may be the same or different.

The (b) nonionic surfactant of ester type may be selected from the group consisting of monounsaturated esters, polyglyceryl diesters, and mixtures thereof.

It may be preferable that the (b) nonionic surfactant of ester type comprise or consist of a mixture of at least one monounsaturated ester and at least one polyglyceryl diester.

The (b) nonionic surfactant of ester type may be a mixture comprising:

i) at least one monounsaturated ester of formula (A)

R¹—C(O)—O—R² (A)

wherein

R¹ and R² represent, respectively, a C₁₈ to C₄₄ fatty chain, at least one of R¹ or R² is monounsaturated;

ii) at least one polyglyceryl diester of formula (B)

R³—C(O)—(O—CH₂—CH(OH)—CH₂)ₙ—O—C(O)—R⁴ (B)

wherein

R³ and R⁴ represent, respectively, a saturated C₁₈ to C₄₄ fatty chain, linear or branched, and n is an integer between 2 and 6; and

iii) at least one C₁₀—C₃₀ fatty alcohol.

According to an embodiment, in the formula (A), R¹ and R² represent, respectively, a C₁₈-C₄₀ fatty chain, more preferably a C₁₈-C₃₀ fatty chain. At least one of R¹ or R² is monounsaturated.

More specifically, in formula (A), the R¹—C(O) group corresponds to the residue of a fatty acid. The R¹ may be linear or monounsaturated, and comprises at least 18 carbon atoms. Mention can be made of oleic (C_{18:1}), gadoleic (C_{20:1}), erucic (C_{22:1}) acid, up to hexaconenoic (C_{26:1}) acid as unsaturated acids. The R¹—C(O) group may also be derived from branched and saturated acids of at least 18 carbon atoms, also called Guerbet acids. The R²—O—group may be derived from monounsaturated linear fatty alcohols with at least 18 carbon atoms. Mention can therefore be made of octadecenol, eicosenol, docosenol and hexacosenol. The carbon chain of the alcohol may also be branched and saturated and comprise at least 18 carbon atoms. Such alcohols are also called Guerbet alcohols.

Preferably, the monounsaturated ester of formula (A) is a mixture of esters comprising various lengths of fatty chains in their structures. More preferably, such a monounsaturated ester is liquid at ambient temperature.

A preferred monounsaturated ester that can be mentioned is, for example, the product commonly called jojoba oil (or jojoba esters), the liquid nature being due to the presence of monounsaturated chains. This oil comprises in particular C_{18:1} (preferably minority), C_{20:1} and C_{22:1} (preferably majority with C_{20:1}>C_{22:1}) unsaturated fatty acid esters, with C_{20:1}, C_{22:1} and C_{24:1} unsaturated fatty alcohols.

According to an embodiment, in formula (B), the R³—C(O)— group corresponds to the residue of a C₁₈ to C₄₄ fatty acid, said acid usually being linear and saturated, and preferably corresponds to a linear and saturated C₂₀ to C₃₄ fatty acid. This therefore includes eicosanoic (or arachidic) acid (C₂₀), docosanoic (or behenic) acid (C₂₂), tetracosanoic (or lignoceric) acid (C₂₄), and hexacosanoic (or cerotic) acid (C₂₆). The R⁴ group corresponds to the hydrocarbon chain of the alcohol, said alcohol usually being saturated and linear and having a C₁₈ to C₄₄ chain, preferably C₂₀ to C₃₄ chain. n is an integer between 2 and 6, preferably from 2 and 4, and more preferably 3.

According to the present invention, the polyglyceryl diester may be obtained by esterification of a solid wax in the presence of at least one polyol.

Waxes have a complex composition. They have the common feature of containing a mixture of acid monoesters and very long chain fatty alcohols such as C₁₀-C₃₀ fatty alcohols.

Depending on the source of the wax, the mixture of monoesters may also contain a certain proportion of hydroxyacid esters such as hydroxypalmitic or hydroxystearic acid. This is the case, for example, for beeswax. Preferably said alcohol is eicosanol, docosanol or tetracosanol. Beeswax, carnauba wax, candelilla wax, rice bran wax, sunflower wax, ouricury wax, Shellac wax and sugarcane wax are examples of natural solid waxes. Preferably, the solid wax is beeswax.

Solid waxes suitable for obtaining the polyglyceryl diester have a melting point between 50 and 90° C. They correspond to mixtures essentially comprising monoesters having the formula R¹—C(O)—O—R², where the R¹—C(O)— group corresponds to the residue of a fatty acid, said acid usually being linear and saturated and having a number of carbon atoms of at least 18, and in particular 20, and preferably up to 44 and more preferably 34. This therefore includes eicosanoic (or arachidic) acid (C₂₀), docosanoic (or behenic) acid (C₂₂), tetracosanoic (or lignoceric) acid (C₂₄), and hexacosanoic (or cerotic) acid (C₂₆). Depending on the source of the wax, the mixture of monoesters may also contain a certain proportion of hydroxyacid esters such as hydroxypalmitic or hydroxystearic acid. This is the case, for example, for beeswax. The R² group corresponds to the hydrocarbon chain of the alcohol, said alcohol usually being saturated and linear and having a number of carbon atoms of at least 18, and in particular 20, preferably up to 44 and more preferably 34. Preferably said alcohol is eicosanol, docosanol or tetracosanol. Beeswax, carnauba wax, candelilla wax, rice bran wax, sunflower wax, ouricury wax, Shellac wax and sugarcane wax are examples of natural solid waxes.

Preferably, the solid wax suitable for the esterification reaction is beeswax.

Preferably, the polyol used for esterification is selected from the group comprising ethylene glycol, diethylene glycol, triethylene glycol, 2-methyl propanediol, propylene glycol, butylene glycol, neopentyl glycol, hexylene glycol, octylene glycol, polyethylene glycol, polypropylene glycol, trimethylol propane, sorbitol, erythritol, pentaerythritol, dipentaerythritol, glycerol, diglycerol and polyglycerol (i.e. a polymer of glycerol units). More preferably, the polyol is a polyglycerol, having an average degree of polymerization between 2 and 6, preferably of 3.

Preferably, the polyol is polyglycerol-3.

Preferably, the (b) nonionic surfactant of ester type is a wax derivative obtained by reacting together at least one solid wax and at least one monounsaturated ester of formula (A) in the presence of at least one polyol and optionally at least one catalyst. In such a case, a transesterification reaction occurs between the various chemical entities yielding the wax derivative.

The preferred catalysts are hydroxides or alkaline or alkaline earth alkoxides, calcium hydroxide, potassium or sodium carbonates or catalysts based on tin or titanium.

Preferably, the solid wax is advantageously selected from the group comprising carnauba wax, candelilla wax, rice bran wax, sunflower wax, sugarcane wax, ouricury wax, beeswax and

Shellac wax.

In a preferred embodiment, the wax derivative is obtained by reacting jojoba oil (also called as jojoba wax), beeswax and a polyglycerol, such as polyglycerol-3.

In practice, the reaction is preferably conducted at a temperature of between 100° C. and 220° C., advantageously between 150° C. and 200° C. Preferably, the monounsaturated ester/solid wax mass ratio varies between 5/95 and 95/5, and advantageously between 30/70 and 75/25. The esters of formula (A) and (B)/polyol mass ratio preferably varies between 1/99 and 99/1, and advantageously between 95/5 and 50/50. Preferably, the proportion of esterified polyol represents between 0.5 and 50% by weight of the mixture, the proportion of esterified fatty acids represents between 20 and 60% by weight of the mixture, and the proportion of esterified fatty alcohols represents between 20 and 60% by weight of the mixture.

The (b) nonionic surfactant of ester type may further comprise a diester of a C₁₄-C₂₂ fatty acid with a polyglycerol.

Typically, the C₁₄-C₂₂ fatty acid may be chosen from the group of myristic acid, stearic acid, isostearic acid, palmitic acid, oleic acid, behenic acid, erucic acid and arachidic acid, and mixtures thereof.

The polyglycerol may be a polymer of glycerol units, preferably a polymer having an average degree of polymerization between 4 and 8, preferably of 6.

Preferably, said diester is a diester of distearic acid with hexaglycerol. Preferably, it is polyglyceryl-6 distearate.

The (b) nonionic surfactant of ester type may further comprise a fatty alcohol containing from 10 to 30 carbon atoms.

As examples of fatty alcohols that may be used, mention may be made of linear or branched fatty alcohols, of synthetic origin or alternatively of natural origin, for instance alcohols originating from vegetable material (coconut, palm kernel, palm, etc.) or animal material (tallow, etc.). Use is preferably made of a fatty alcohol comprising from 20 to 26 carbon atoms, preferably from 10 to 24 carbon atoms and more preferentially from 12 to 22 carbon atoms.

As particular examples of fatty alcohols that may be used in the context of the present invention, mention may in particular be made of lauryl alcohol, myristyl alcohol, cetyl alcohol, stearyl alcohol, isostearyl alcohol, palmityl alcohol, oleyl alcohol, cetearyl alcohol (mixture of cetyl alcohol and stearyl alcohol), behenyl alcohol, erucyl alcohol and arachidyl alcohol, and mixtures thereof.

It may be preferable that the (b) nonionic surfactant of ester type be a mixture comprising at least one monounsaturated ester of formula (A) wherein R¹ and R² represent, respectively, a C₁₈-C₃₀ fatty chain, and at least one of R¹ or R² is monounsaturated; at least one polyglyceryl diester of formula (B) wherein, R³ and R⁴ each represent a saturated C₂₀ to C₃₄ fatty chain, linear or branched; and cetyl alcohol.

It may be more preferable that the (b) nonionic surfactant of ester type be a mixture of at least an ester obtained by esterification of a solid wax with a polyol, of a fatty acid diester with a polyglycerol, of a jojoba wax (preferably a jojoba wax ester), and of a fatty alcohol. Said ester is non-ionic.

In addition, it is particularly advantageous, according to the present invention, to use together a mixture of polyglyceryl-6 distearate and polyglyceryl-3 beeswax, with cetyl alcohol and jojoba wax. Among the mixtures that are particularly preferred, mention may be made of the product sold by the company Gattefosse under the name Emulium® Mellifera, comprising jojoba wax, cetyl alcohol, polyglyceryl-6 distearate, and polyglyceryl-3 beeswax (INCI name: Polyglyceryl-6 Distearate (and) Jojoba Esters (and) Polyglyceryl-3 Beeswax (and) Cetyl Alcohol). Said mixture comprises from 5 to 30% by weight of the total weight of the mixture of jojoba wax; from 3 to 15% by weight of cetyl alcohol; at least 50% by weight of polyglyceryl-6 distearate; and from 3 to 15% by weight of polyglyceryl-3 beeswax.

The (b) nonionic surfactant of ester type may be a mixture of polyglyceryl-6 distearate, jojoba esters, polyglyceryl-3 beeswax, and cetyl alcohol.

The amount of the (b) nonionic surfactant of ester type in the composition according to the present invention may be 0.05% by weight or more, preferably 0.1% by weight or more, and more preferably 0.5% by weight or more, relative to the total weight of the composition.

The amount of the (b) nonionic surfactant of ester type in the composition according to the present invention may be 10% by weight or less, preferably 5% by weight or less, and more preferably 3% by weight or less, relative to the total weight of the composition.

The amount of the (b) nonionic surfactant of ester type in the composition according to the present invention may be from 0.05% to 10% by weight, preferably from 0.1 to 5% by weight, and more preferably from 0.5% to 3% by weight, relative to the total weight of the composition.

(Alkylpolyglycoside Type Surfactant)

The composition according to the present invention comprises (c) at least one surfactant of alkylpolyglycoside type. If two or more surfactants of alkylpolyglycoside type are used, they may be the same or different.

For the purposes of the present invention, the term “alkylpolyglycoside” is intended to mean an alkylmonosaccharide (degree of polymerization 1) or an alkylpolysaccharide (degree of polymerization greater than 1).

The alkylpolyglycosides may be used alone or in the form of mixtures of several alkylpolyglycosides.

The (c) surfactant of alkylpolyglycoside type may be selected from the compound of formula (I):

R(O)(G)ₓ (I)

in which the radical R is a linear or branched C₁₂-C₂₂ alkyl radical, preferably a C₁₂-C₂₀ alkyl radical, G is a saccharide residue and x ranges from 1 to 5, preferably from 1.05 to 2.5 and more preferentially from 1.1 to 2.

The saccharide residue may be chosen from the group of glucose, dextrose, saccharose, fructose, galactose, maltose, maltotriose, lactose, cellobiose, mannose, ribose, dextran, talose, allose, xylose, levoglucan, cellulose and starch. More preferentially, the saccharide residue denotes glucose.

It should also be noted that each unit of the polysaccharide part of the alkylpolyglycoside may be in a or 13 isomer form, in L or D form, and the configuration of the saccharide residue may be of furanoside or pyranoside type.

It is, of course, possible to use mixtures of alkylpolysaccharides, which may differ from each other in the nature of the borne alkyl unit and/or the nature of the bearing polysaccharide chain.

In addition, it is particularly advantageous, according to the present invention, to use together a fatty alcohol and an alkylpolyglycoside of which the alkyl part is identical to that of the selected fatty alcohol.

Fatty alcohol/alkylpolyglycoside emulsifying mixtures as defined are described in particular in patent applications WO 92/06778, WO 95/13863 and WO 98/47610.

Among the fatty alcohol/alkylpolyglycoside mixtures that are particularly preferred, mention may be made of the products sold by the company SEPPIC under the name Montanov®, such as the following mixtures:
cetylstearyl alcohol/cocoyl glucoside (Montanov 82®),
arachidyl alcohol and behenyl alcohol/arachidyl glucoside (Montanov 802®),
myristyl alcohol/myristyl glucoside (Montanov 14®),
cetylstearyl alcohol/cetylstearyl glucoside (Montanov 68®),
C₁₄-C₂₂ alcohol/C₁₂-C₂₀ alkyl glucoside (Montanov L®),
cocoyl alcohol/cocoyl glucoside (Montanov S®) and
isostearyl alcohol/isostearyl glucoside (Montanov WO 18®).

According to one particular embodiment, the alkylpolyglycoside used in a composition according to the present invention is C₁₂-C₂₀glucoside. It is advantageously used as a mixture with a C₁₄-C₂₂ alcohol.

According to one particular embodiment of the present invention, use is thus made of the C₁₄-C₂₂ alcohol/C₁₂-C₂₀ alkylglucoside mixture, such as the product sold by the company SEPPIC under the name Montanov 68®, consisting of approximately 20% of C₁₂-C₂₀ alkylglucoside and of approximately 80% of C₁₄-C₂₂ alcohol.

The amount of the (c) surfactant of alkylpolyglycoside type in the composition according to the present invention may be 0.01% by weight or more, preferably 0.03% by weight or more, and more preferably 0.05% by weight or more, relative to the total weight of the composition.

The amount of the (c) surfactant of alkylpolyglycoside type in the composition according to the present invention may be 5% by weight or less, preferably 1% by weight or less and more preferably 0.5% by weight or less, relative to the total weight of the composition.

The amount of the (c) surfactant of alkylpolyglycoside type in the composition according to the present invention may be from 0.01% to 5% by weight, preferably from 0.03% to 1% by weight, and more preferably from 0.05% to 0.5% by weight, relative to the total weight of the composition.

(Hydrophilic Gelling Agent)

The composition according to the present invention comprises (d) at least one polysaccharide hydrophilic gelling agent other than microcrystalline cellulose. If two or more polysaccharide hydrophilic gelling agents are used, they may be the same or different.

The polysaccharide hydrophilic gelling agent(s) may be selected from glucans, modified and unmodified starches (such as those obtained, for example, from cereals such as wheat, maize or rice, from vegetables such as haricot beans, from root crops such as potatoes or cassava), amylose, amylopectin, glycogen, dextrans, celluloses and derivatives thereof (methylcelluloses, hydroxyalkylcelluloses, hydroxyethylcelluloses, hydroxypropylcelluloses, carboxymethylcelluloses), mannans, xylans, lignins, arabans, galactans, galacturonans, chitin, chitosans, glucuronoxylans, arabinoxylans, xyloglucans, glucomannans, pectic acids and pectins, alginic acid and alginates, arabinogalactans, carrageenans, agars, glycosaminoglucans, gums arabic, gums tragacanth, gums ghatti, karaya gums, carob gums, galactomannans such as guar gums and their nonionic derivatives (hydroxypropylguar) and xanthan gums, and mixtures thereof.

Generally speaking, the compounds of this type that can be used in the present invention are selected from those described in particular in Encyclopedia of Chemical Technology, Kirk-Othmer, Third Edition, 1982, Volume 3, pp. 896-900 and Volume 15, pp. 439-58, in Polymers in Nature by E. A. MacGregor and C. T. Greenwood, John Wiley & Sons, Chapter 6, pp. 240-328, 1980, and in Industrial Gums--polysaccharides and their derivatives, edited by Roy L. Whistler, Second Edition, Academic Press Inc., the content of these three works being included in their entirety in the present patent application by way of reference.

The (d) polysaccharide hydrophilic gelling agent is selected from xanthan gum, carob gum and mixtures thereof, preferably carob gum.

It is preferable that the (d) polysaccharide hydrophilic gelling agent be a mixture of xanthan gum and carob gum, for example NOMCORT® CG (Nisshin Oillio Group, Ltd.) comprising the xanthan gum and the ceratonia siliqua (carob) gum in a ratio of 50/50 by weight.

The amount of the (d) polysaccharide hydrophilic gelling agent in the composition according to the present invention may be 0.0005% by weight or more, preferably 0.0008% by weight or more, and more preferably 0.001% by weight or more, relative to the total weight of the composition.

The amount of the (d) polysaccharide hydrophilic gelling agent in the composition according to the present invention may be 0.5% by weight or less, preferably 0.2% by weight or less and more preferably 0.1% by weight or less, relative to the total weight of the composition.

The amount of the (d) polysaccharide hydrophilic gelling agent in the composition according to the present invention may be from 0.0005% to 0.5% by weight, preferably from 0.0008% to 0.2% by weight, and more preferably from 0.001% to 0.1% by weight, relative to the total weight of the composition.

(Oil)

The composition according to the present invention comprises (e) at least one oil. If two or more oils are used, they may be the same or different.

Here, “oil” means a fatty compound or substance which is in the form of a liquid or a paste (non-solid) at room temperature (25° C.) under atmospheric pressure (760 mmHg). As the oils, those generally used in cosmetics can be used alone or in combination thereof. These oils may be volatile or non-volatile.

The oil may be a non-polar oil such as a hydrocarbon oil, a silicone oil, or the like; a polar oil such as a plant or animal oil and an ester oil or an ether oil; or a mixture thereof.

The oil may be selected from the group consisting of oils of plant or animal origin, synthetic oils, silicone oils, hydrocarbon oils and fatty alcohols.

As examples of plant oils, mention may be made of, for example, linseed oil, camellia oil, macadamia nut oil, corn oil, mink oil, olive oil, avocado oil, sasanqua oil, castor oil, safflower oil, jojoba oil, sunflower oil, almond oil, rapeseed oil, sesame oil, soybean oil, peanut oil, and mixtures thereof.

As examples of animal oils, mention may be made of, for example, squalene and squalane.

As examples of synthetic oils, mention may be made of alkane oils such as isododecane and isohexadecane, ester oils, ether oils, and artificial triglycerides.

The ester oils are preferably liquid esters of saturated or unsaturated, linear or branched C₁-C₂₆ aliphatic monoacids or polyacids and of saturated or unsaturated, linear or branched C₁-C₂₆ aliphatic monoalcohols or polyalcohols, the total number of carbon atoms of the esters being greater than or equal to 10.

Preferably, for the esters of monoalcohols, at least one from among the alcohol and the acid from which the esters of the present invention are derived is branched.

Among the monoesters of monoacids and of monoalcohols, mention may be made of ethyl palmitate, ethyl hexyl palmitate, isopropyl palmitate, dicaprylyl carbonate, alkyl myristates such as isopropyl myristate or ethyl myristate, isocetyl stearate, 2-ethylhexyl isononanoate, isononyl isononanoate, isodecyl neopentanoate and isostearyl neopentanoate.

Esters of C₄-C₂₂ dicarboxylic or tricarboxylic acids and of C₁-C₂₂ alcohols, and esters of monocarboxylic, dicarboxylic or tricarboxylic acids and of non-sugar C₄-C₂₆ dihydroxy, trihydroxy, tetrahydroxy or pentahydroxy alcohols may also be used.

Mention may especially be made of: diethyl sebacate; isopropyl lauroyl sarcosinate; diisopropyl sebacate; bis(2-ethylhexyl) sebacate; diisopropyl adipate; di-n-propyl adipate; dioctyl adipate; bis(2-ethylhexyl) adipate; diisostearyl adipate; bis(2-ethylhexyl) maleate; triisopropyl citrate; triisocetyl citrate; triisostearyl citrate; glyceryl trilactate; glyceryl trioctanoate; trioctyldodecyl citrate; trioleyl citrate; neopentyl glycol diheptanoate; diethylene glycol diisononanoate.

As ester oils, one can use sugar esters and diesters of C₆-C₃₀ and preferably C₁₂-C₂₂ fatty acids. It is recalled that the term “sugar” means oxygen-bearing hydrocarbon-based compounds containing several alcohol functions, with or without aldehyde or ketone functions, and which comprise at least 4 carbon atoms. These sugars may be monosaccharides, oligosaccharides or polysaccharides.

Examples of suitable sugars that may be mentioned include sucrose (or saccharose), glucose, galactose, ribose, fucose, maltose, fructose, mannose, arabinose, xylose and lactose, and derivatives thereof, especially alkyl derivatives, such as methyl derivatives, for instance methylglucose.

The sugar esters of fatty acids may be chosen especially from the group comprising the esters or mixtures of esters of sugars described previously and of linear or branched, saturated or unsaturated C₆-C₃₀ and preferably C₁₂-C₂₂ fatty acids. If they are unsaturated, these compounds may have one to three conjugated or non-conjugated carbon-carbon double bonds.

The esters according to this variant may also be selected from monoesters, diesters, triesters, tetraesters and polyesters, and mixtures thereof.

These esters may be, for example, oleates, laurates, palmitates, myristates, behenates, cocoates, stearates, linoleates, linolenates, caprates and arachidonates, or mixtures thereof such as, especially, oleopalmitate, oleostearate and palmitostearate mixed esters, as well as pentaerythrityl tetraethyl hexanoate.

More particularly, use is made of monoesters and diesters and especially sucrose, glucose or methylglucose monooleates or dioleates, stearates, behenates, oleopalmitates, linoleates, linolenates and oleostearates.

An example that may be mentioned is the product sold under the name Glucate® DO by the company Amerchol, which is a methylglucose dioleate.

As examples of preferable ester oils, mention may be made of, for example, diisopropyl adipate, dioctyl adipate, 2-ethylhexyl hexanoate, ethyl laurate, cetyl octanoate, octyldodecyl octanoate, isodecyl neopentanoate, myristyl propionate, 2-ethylhexyl 2-ethylhexanoate, 2-ethylhexyl octanoate, 2-ethylhexyl caprylate/caprate, methyl palmitate, ethyl palmitate, isopropyl palmitate, dicaprylyl carbonate, isopropyl lauroyl sarcosinate, isononyl isononanoate, ethylhexyl palmitate, isohexyl laurate, hexyl laurate, isocetyl stearate, isopropyl isostearate, isopropyl myristate, isodecyl oleate, glyceryl tri(2-ethylhexanoate), pentaerythrityl tetra(2-ethylhexanoate), 2-ethylhexyl succinate, diethyl sebacate, and mixtures thereof.

As examples of artificial triglycerides, mention may be made of, for example, capryl caprylyl glycerides, glyceryl trimyristate, glyceryl tripalmitate, glyceryl trilinolenate, glyceryl trilaurate, glyceryl tricaprate, glyceryl tricaprylate, glyceryl tri(caprate/caprylate) (INCI name: Caprylic/Capric Triglyceride) and glyceryl tri(caprate/caprylate/linolenate).

As examples of silicone oils, mention may be made of, for example, linear organopolysiloxanes such as dimethylpolysiloxane (INCI name: Dimethicone), methylphenylpolysiloxane, methylhydrogenpolysiloxane, and the like; cyclic organopolysiloxanes such as cyclohexasiloxane, octamethylcyclotetrasiloxane, decamethylcyclopentasiloxane, dodecamethylcyclohexasiloxane, and the like; and mixtures thereof.

Preferably, silicone oil is chosen from liquid polydialkylsiloxanes, especially liquid polydimethylsiloxanes (PDMS) and liquid polyorganosiloxanes comprising at least one aryl group.

These silicone oils may also be organomodified. The organomodified silicones that can be used according to the present invention are silicone oils as defined above and comprise in their structure one or more organofunctional groups attached via a hydrocarbon-based group.

Organopolysiloxanes are defined in greater detail in Walter Noll's Chemistry and Technology of Silicones (1968), Academic Press. They may be volatile or non-volatile.

When they are volatile, the silicones are more particularly chosen from those having a boiling point of between 60° C. and 260° C., and even more particularly from:

(i) cyclic polydialkylsiloxanes comprising from 3 to 7 and preferably 4 to 5 silicon atoms. These are, for example, octamethylcyclotetrasiloxane sold in particular under the name Volatile Silicone® 7207 by Union Carbide or Silbione® 70045 V2 by Rhodia, decamethylcyclopentasiloxane sold under the name Volatile Silicone® 7158 by Union Carbide, Silbione® 70045 V5 by Rhodia, and dodecamethylcyclopentasiloxane sold under the name Silsoft 1217 by Momentive Performance Materials, and mixtures thereof. Mention may also be made of cyclocopolymers of the type such as dimethylsiloxane/methylalkylsiloxane, such as Silicone Volatile® FZ 3109 sold by the company Union Carbide, of formula:

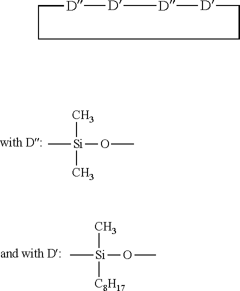

Mention may also be made of mixtures of cyclic polydialkylsiloxanes with organosilicon compounds, such as the mixture of octamethylcyclotetrasiloxane and tetratrimethylsilylpentaerythritol (50/50) and the mixture of octamethylcyclotetrasiloxane and oxy-1,1′-bis(2,2,2′,2′,3,3′-hexatrimethylsilyloxy)neopentane; and

(ii) linear volatile polydialkylsiloxanes containing 2 to 9 silicon atoms and having a viscosity of less than or equal to 5×10⁻⁶ m²/s at 25° C. An example is decamethyltetrasiloxane sold in particular under the name SH 200 by the company Toray Silicone. Silicones belonging to this category are also described in the article published in Cosmetics and Toiletries, Vol. 91, Jan. 76, pp. 27-32, Todd & Byers, Volatile Silicone Fluids for Cosmetics. The viscosity of the silicones is measured at 25° C. according to ASTM standard 445 Appendix C.

Non-volatile polydialkylsiloxanes may also be used. These non-volatile silicones are more particularly chosen from polydialkylsiloxanes, among which mention may be made mainly of polydimethylsiloxanes containing trimethylsilyl end groups.

Among these polydialkylsiloxanes, mention may be made, in a non-limiting manner, of the following commercial products:
the Silbione® oils of the 47 and 70 047 series or the Mirasil® oils sold by Rhodia, for instance the oil 70 047 V 500 000;
the oils of the Mirasil® series sold by the company Rhodia;
the oils of the 200 series from the company Dow Corning, such as DC₂₀₀ with a viscosity of 60 000 mm²/s; and
the Viscasil® oils from General Electric and certain oils of the SF series (SF 96, SF 18) from General Electric.

Mention may also be made of polydimethylsiloxanes containing dimethylsilanol end groups known under the name dimethiconol (CTFA), such as the oils of the 48 series from the company Rhodia.

Among the silicones containing aryl groups, mention may be made of polydiarylsiloxanes, especially polydiphenylsiloxanes and polyalkylarylsiloxanes such as phenyl silicone oil.

The phenyl silicone oil may be chosen from the phenyl silicones of the following formula:

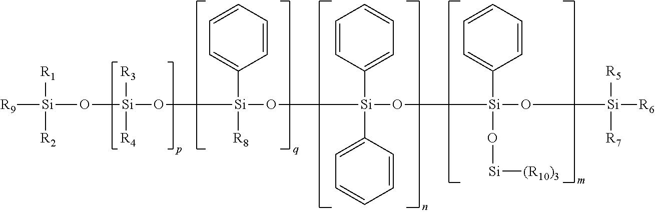

in which

R₁ to R₁₀, independently of each other, are saturated or unsaturated, linear, cyclic or branched C₁-C₃₀ hydrocarbon-based radicals, preferably C₁-C₁₂ hydrocarbon-based radicals, and more preferably C₁-C₆ hydrocarbon-based radicals, in particular methyl, ethyl, propyl or butyl radicals, and

m, n, p and q are, independently of each other, integers between 0 and 900 inclusive, preferably 0 and 500 inclusive, and more preferably 0 and 100 inclusive, with the proviso that the sum n+m+q is other than 0.

Examples that may be mentioned include the products sold under the following names:
the Silbione® oils of the 70 641 series from Rhodia;
the oils of the Rhodorsil® 70 633 and 763 series from Rhodia;
the oil Dow Corning 556 Cosmetic Grade Fluid from Dow Corning;
the silicones of the PK series from Bayer, such as the product PK20;
certain oils of the SF series from General Electric, such as SF 1023, SF 1154, SF 1250 and SF 1265.

As the phenyl silicone oil, phenyl trimethicone (R₁ to R₁₀ are methyl; p, q, and n=0; m=1 in the above formula) is preferable.

The organomodified liquid silicones may especially contain polyethyleneoxy and/or polypropyleneoxy groups. Mention may thus be made of the silicone KF-6017 proposed by Shin-Etsu, and the oils Silwet® L722 and L77 from the company Union Carbide.

Hydrocarbon oils may be chosen from:
linear or branched, optionally cyclic, C₆-C₁₆ lower alkanes. Examples that may be mentioned include hexane, undecane, dodecane, tridecane, and isoparaffins, for instance isohexadecane, isododecane and isodecane; and
linear or branched hydrocarbons containing more than 16 carbon atoms, such as liquid paraffins, liquid petroleum jelly, polydecenes and hydrogenated polyisobutenes such as Parleam®, and squalane.

As preferable examples of hydrocarbon oils, mention may be made of, for example, linear or branched hydrocarbons such as isohexadecane, isododecane, squalane, mineral oil (e.g., liquid paraffin), paraffin, vaseline or petrolatum, naphthalenes, and the like; hydrogenated polyisobutene, isoeicosan, and decene/butene copolymer; and mixtures thereof.

The term “fatty” in the fatty alcohol means the inclusion of a relatively large number of carbon atoms. Thus, alcohols which have 4 or more, preferably 6 or more, and more preferably 12 or more carbon atoms are encompassed within the scope of fatty alcohols. The fatty alcohol may be saturated or unsaturated. The fatty alcohol may be linear or branched.

The fatty alcohol may have the structure R—OH wherein R is chosen from saturated and unsaturated, linear and branched radicals containing from 4 to 40 carbon atoms, preferably from 6 to 30 carbon atoms, and more preferably from 12 to 20 carbon atoms. In at least one embodiment, R may be chosen from C₁₂-C₂₀ alkyl and C₁₂-C₂₀ alkenyl groups. R may or may not be substituted with at least one hydroxyl group.

As examples of the fatty alcohol, mention may be made of lauryl alcohol, isostearyl alcohol, undecylenyl alcohol, octyldodecanol, hexyldecanol, oleyl alcohol, linoleyl alcohol, palmitoleyl alcohol, arachidonyl alcohol, and mixtures thereof.

Thus, the fatty alcohol may be selected from straight or branched, saturated or unsaturated C₆-C₃₀ alcohols, preferably straight or branched, saturated C₆-C₃₀ alcohols, and more preferably straight or branched, saturated C₁₂-C₂₀ alcohols.

The term “saturated fatty alcohol” here means an alcohol having a long aliphatic saturated carbon chain. It is preferable that the saturated fatty alcohol be selected from any linear or branched, saturated C₆-C₃₀ fatty alcohols. Among the linear or branched, saturated C₆-C₃₀ fatty alcohols, linear or branched, saturated C₁₂-C₂₀ fatty alcohols may preferably be used. Any linear or branched, saturated C₁₆-C₂₀ fatty alcohols may be more preferably used. Branched C₁₆-C₂₀ fatty alcohols may be even more preferably used.

As examples of saturated fatty alcohols, mention may be made of lauryl alcohol, isostearyl alcohol, undecylenyl alcohol, octyldodecanol, hexyldecanol, and mixtures thereof. In one embodiment, octyldodecanol andhexyldecanol can be used as a saturated fatty alcohol.

According to at least one embodiment, the fatty alcohol used in the composition according to the present invention is preferably chosen from octyldodecanol, hexyldecanol and mixtures thereof.

The amount of the (e) oil in the composition according to the present invention may be 0.1% by weight or more, preferably 0.5% by weight or more, and more preferably 1% by weight or more, relative to the total weight of the composition.

The amount of the (e) oil in the composition according to the present invention may be 20% by weight or less, preferably 10% by weight or less and more preferably 8% by weight or less, relative to the total weight of the composition.

The amount of the (e) oil in the composition according to the present invention may be from 0.1% to 20% by weight, preferably from 0.5% to 10% by weight, and more preferably from 1% to 8% by weight, relative to the total weight of the composition.

(Water)

The composition according to the present invention includes (f) water.

The amount of the (f) water in the composition according to the present invention may be 50% by weight or more, preferably 55% by weight or more, and more preferably 60% by weight or more, relative to the total weight of the composition.

The amount of the (f) water in the composition according to the present invention may be 95% by weight or less, preferably 90% by weight or less and more preferably 85% by weight or less, relative to the total weight of the composition.

The amount of the (f) water in the composition according to the present invention may be from 50% to 95% by weight, preferably from 55% to 90% by weight, and more preferably from 60% to 85% by weight, relative to the total weight of the composition.

The (f) water can form an aqueous phase of the composition according to the present invention, if the composition is of the O/W type, in particular in the form of an O/W emulsion.

(Anionic Surfactant)

The composition according to the present invention may comprise (g) at least one anionic surfactant. If two or more anionic surfactants are used, they may be the same or different.

The (g) anionic surfactant may be useful in enhancing the stability of the composition according to the present invention.

The (g) anionic surfactants may be chosen in particular from anionic derivatives of proteins of vegetable origin or of silk proteins, phosphates and alkyl phosphates, carboxylates, sulphosuccinates, amino acid derivatives, alkyl sulphates, alkyl ether sulphates, sulphonates, isethionates, taurates, alkyl sulphoacetates, polypeptides, anionic derivatives of alkyl polyglucosides, and their mixtures.

1) Anionic derivatives of proteins of vegetable origin are protein hydrolysates comprising a hydrophobic group, it being possible for the said hydrophobic group to be naturally present in the protein or to be added by reaction of the protein and/or of the protein hydrolysate with a hydrophobic compound. The proteins are of vegetable origin or derived from silk, and the hydrophobic group can in particular be a fatty chain, for example an alkyl chain comprising from 10 to 22 carbon atoms. Mention may more particularly be made, as anionic derivatives of proteins of vegetable origin, of apple, wheat, soybean or oat protein hydrolysates comprising an alkyl chain having from 10 to 22 carbon atoms, and their salts. The alkyl chain can in particular be a lauryl chain and the salt can be a sodium, potassium and/or ammonium salt.

Thus, mention may be made, as protein hydrolysates comprising a hydrophobic group, for example, of salts of protein hydrolysates where the protein is a silk protein modified by lauric acid, such as the product sold under the name Kawa Silk by Kawaken; salts of protein hydrolysates where the protein is a wheat protein modified by lauric acid, such as the potassium salt sold under the name Aminofoam W OR by Croda (CTFA name: potassium lauroyl wheat amino acids) and the sodium salt sold under the name Proteol LW 30 by Seppic (CTFA name: sodium lauroyl wheat amino acids); salts of protein hydrolysates where the protein is an oat protein comprising an alkyl chain having from 10 to 22 carbon atoms and more especially salts of protein hydrolysates where the protein is an oat protein modified by lauric acid, such as the sodium salt sold under the name Proteol OAT (30% aqueous solution) by Seppic (CTFA name: sodium lauroyl oat amino acids); or salts of apple protein hydrolysates comprising an alkyl chain having from 10 to 22 carbon atoms, such as the sodium salt sold under the name Proteol APL (30% aqueous/glycol solution) by Seppic (CTFA name: sodium cocoyl apple amino acids). Mention may also be made of the mixture of lauroyl amino acids (aspartic acid, glutamic acid, glycine, alanine) neutralized with sodium N-methylglycinate sold under the name Proteol SAV 50 S by Seppic (CTFA name: sodium cocoyl amino acids).

2) Mention may be made, as phosphates and alkyl phosphates, for example, of monoalkyl phosphates and dialkyl phosphates, such as lauryl monophosphate, sold under the name MAP 20® by Kao Chemicals, the potassium salt of dodecyl phosphate, the mixture of mono- and diesters (predominantly diester) sold under the name Crafol AP-31® by Cognis, the mixture of octyl phosphate monoester and diester, sold under the name Crafol AP-20® by Cognis, the mixture of ethoxylated (7 mol of EO) 2-butyloctyl phosphate monoester and diester, sold under the name Isofol 12 7 EO-Phosphate Ester® by Condea, the potassium or triethanolamine salt of mono(C₁₂-C₁₃)alkyl phosphate, sold under the references Arlatone MAP 230K-40® and Arlatone MAP 230T-60® by Uniqema, potassium lauryl phosphate, sold under the name Dermalcare MAP XC-99/09® by Rhodia Chimie, and potassium cetyl phosphate, sold under the name AMPHISOL K by DSM NUTRITIONAL PRODUCTS.

3) Mention may be made, as carboxylates, of:
amido ether carboxylates (AEC), such as sodium lauryl amido ether carboxylate (3 EO), sold under the name Akypo Foam 30® by Kao Chemicals;
polyoxyethylenated carboxylic acid salts, such as oxyethylenated (6 EO) sodium lauryl ether carboxylate (65/25/10 C₁₂-C₁₄-C₁₆), sold under the name Akypo Soft 45 NV® by Kao Chemicals, polyoxyethylenated and carboxymethylated fatty acids originating from olive oil, sold under the name Olivem 400® by Biologia E Tecnologia, or oxyethylenated (6 EO) sodium tridecyl ether carboxylate, sold under the name Nikkol ECTD-6NEX® by Nikkol; and
salts of fatty acids (soaps) having a C₆ to C₂₂ alkyl chain which are neutralized with an organic or inorganic base, such as potassium hydroxide, sodium hydroxide, triethanolamine, N-methylglucamine, lysine and arginine.

4) Mention may in particular be made, as amino acid derivatives, of alkali salts of amino acids, such as:
sarcosinates, such as sodium lauroyl sarcosinate, sold under the name Sarkosyl NL 97® by Ciba or sold under the name Oramix L 30® by Seppic, sodium myristoyl sarcosinate, sold under the name Nikkol Sarcosinate MN® by Nikkol, or sodium palmitoyl sarcosinate, sold under the name Nikkol Sarcosinate PN® by Nikkol;
alaninates, such as sodium N-lauroyl-N-methylamidopropionate, sold under the name Sodium Nikkol Alaninate LN 30® by Nikkol or sold under the name Alanone ALE® by Kawaken, or triethanolamine N-lauroyl-N-methylalanine, sold under the name Alanone ALTA® by Kawaken;
glutamates, such as triethanolamine monococoyl glutamate, sold under the name Acylglutamate CT-12® by Ajinomoto, triethanolamine lauroyl glutamate, sold under the name Acylglutamate LT-12® by Ajinomoto;
aspartates, such as the mixture of triethanolamine N-lauroyl aspartate and triethanolamine N-myristoyl aspartate, sold under the name Asparack® by Mitsubishi;
glycine derivatives (glycinates), such as sodium N-cocoyl glycinate, sold under the names Amilite GCS-12® and Amilite GCK 12 by Ajinomoto;
citrates, such as the citric monoester of oxyethylenated (9 mol) coco alcohols, sold under the name Witconol EC 1129 by Goldschmidt; and
galacturonates, such as sodium dodecyl D-galactoside uronate, sold by Soliance.

5) Mention may be made, as sulphosuccinates, for example, of oxyethylenated (3 EO) lauryl (70/30 C₁₂/C₁₄) alcohol monosulphosuccinate, sold under the names Setacin 103 Special® and Rewopol SB-FA 30 K 4® by Witco, the disodium salt of a hemisulphosuccinate of C₁₂-C₁₄ alcohols, sold under the name Setacin F Special Paste® by Zschimmer Schwarz, oxyethylenated (2 EO) disodium oleamidosulphosuccinate, sold under the name Standapol SH 135® by Cognis, oxyethylenated (5 EO) lauramide monosulphosuccinate, sold under the name Lebon A-5000® by Sanyo, the disodium salt of oxyethylenated (10 EO.) lauryl citrate monosulphosuccinate, sold under the name Rewopol SB CS 50® by Witco, or ricinoleic monoethanolamide monosulphosuccinate, sold under the name Rewoderm S 1333® by Witco. Use may also be made of polydimethylsiloxane sulphosuccinates, such as disodium PEG-12 dimethicone sulphosuccinate, sold under the name Mackanate-DC 30 by MacIntyre.

6) Mention may be made, as alkyl sulphates, for example, of triethanolamine lauryl sulphate (CTFA name: TEA lauryl sulphate), such as the product sold by Huntsman under the name Empicol TL40 FL or the product sold by Cognis under the name Texapon T42, which products are at 40% in aqueous solution. Mention may also be made of ammonium lauryl sulphate (CTFA name: ammonium lauryl sulphate), such as the product sold by Huntsman under the name Empicol AL 30FL, which is at 30% in aqueous solution.

7) Mention may be made, as alkyl ether sulphates, for example, of sodium lauryl ether sulphate (CTFA name: sodium laureth sulphate), such as that sold under the names Texapon N40 and Texapon AOS 225 UP by Cognis, or ammonium lauryl ether sulphate (CTFA name: ammonium laureth sulphate), such as that sold under the name Standapol EA-2 by Cognis.

8) Mention may be made, as sulphonates, for example, of α-olefinsulphonates, such as sodium α-olefinsulphonate (C₁₄-C₁₆), sold under the name Bio-Terge AS-40® by Stepan, sold under the names Witconate AOS Protégé® and Sulframine AOS PH 12® by Witco or sold under the name Bio-Terge AS-40 CG® by Stepan, secondary sodium olefinsulphonate, sold under the name Hostapur SAS 30® by Clariant; or linear alkylarylsulphonates, such as sodium xylenesulphonate, sold under the names Manrosol SXS30®, Manrosol SXS40® and Manrosol SXS93® by Manro.

9) Mention may be made, as isethionates, of acylisethionates, such as sodium cocoylisethionate, such as the product sold under the name Jordapon CI P® by Jordan.

10) Mention may be made, as taurates, of the sodium salt of palm kernel oil methyltaurate, sold under the name Hostapon CT Pate® by Clariant; N-acyl-N-methyltaurates, such as sodium N-cocoyl-N-methyltaurate, sold under the name Hostapon LT-SF® by Clariant or sold under the name Nikkol CMT-30-T® by Nikkol, Sodium Methyl Stearoyl Taurate sold under the name Nikkol SMT® or sodium palmitoyl methyltaurate, sold under the name Nikko! PMT® by Nikkol.

11) The anionic derivatives of alkyl polyglucosides can in particular be citrates, tartrates, sulphosuccinates, carbonates and glycerol ethers obtained from alkyl polyglucosides. Mention may be made, for example, of the sodium salt of cocoylpolyglucoside (1,4) tartaric ester, sold under the name Eucarol AGE-ET® by Cesalpinia, the disodium salt of cocoylpolyglucoside (1,4) sulphosuccinic ester, sold under the name Essai 512 MP® by Seppic, or the sodium salt of cocoylpolyglucoside (1,4) citric ester, sold under the name Eucarol AGE-EC® by Cesalpinia.

For the amino acid derivatives, it is preferable that they are chosen from acyl glycine derivatives or glycine derivatives, in particular acyl glycine salt.

The acyl glycine derivatives or glycine derivatives can be chosen from acyl glycine salts (or acyl glycinates) or glycine salts (or glycinates), and in particular from the following.

i) Acyl glycinates of formula (I):

R—HNCH2COOX (I)

in which
R represents an acyl group R′C═O, with R′, which represents a saturated or unsaturated, linear or branched, hydrocarbon chain, preferably comprising from 10 to 30 carbon atoms, more preferably from 12 to 22 carbon atoms, even more preferably from 14 to 22 carbon atoms and better still from 16 to 20 carbon atoms, and
X represents a cation chosen, for example, from the ions of alkali metals, such as Na, Li or K, preferably Na or K, the ions of alkaline earth metals, such as Mg, ammonium groups and their mixtures.

The acyl group can in particular be chosen from the lauroyl, myristoyl, behenoyl, palmitoyl, stearoyl, isostearoyl, olivoyl, cocoyl or oleoyl groups and their mixtures.

Preferably, R is a cocoyl group.

ii) Glycinates of following formula (II):

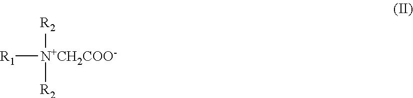

in which:
R₁ represents a saturated or unsaturated, linear or branched, hydrocarbon chain comprising from 10 to 30 carbon atoms, preferably from 12 to 22 carbon atoms and better still from 16 to 20 carbon atoms; R₁ is advantageously chosen from the lauryl, myristyl, palmityl, stearyl, cetyl, cetearyl or oleyl groups and their mixtures and preferably from the stearyl and oleyl groups, and
the R₂ groups, which are identical or different, represent an R″OH group, R″ being an alkyl group comprising from 2 to 10 carbon atoms, preferably from 2 to 5 carbon atoms.

Mention may be made, as the compound of formula (I), for example, of the compounds carrying the INCI name sodium cocoyl glycinate, such as, for example, Amilite GCS-12, sold by Ajinomoto, or potassium cocoyl glycinate, such as, for example, Amilite GCK-12 from Ajinomoto.

Use may be made, as compounds of formula (II), of dihydroxyethyl oleyl glycinate or dihydroxyethyl stearyl glycinate.

Preferably anionic surfactants are not soaps. Thus, preferably anionic surfactants are chosen from synthetic anionic surfactants. More preferably, anionic surfactants are chosen from amido ether carboxylates; alkyl sulfates; alkyl ether sulfates; olefin sulfonates and acylisethionates; and mixtures thereof.

It is preferable that the (g) anionic surfactant be selected from the group consisting of: sodium laureth sulfate, ammonium laureth sulfate, disodium lauryl sulfosuccinate, disodium laureth sulfosuccinate, diammonium lauryl sulfosuccinate, diethylhexyl sodium sulfosuccinate, sodium oleyl succinate, sodium lauroyl methyl isethionate, sodium lauryl isethionate, sodium cocoyl isethionate, sodium laureth-5 carboxylate, lauryl ether carboxylic acid, ammonium lauryl sulfate, triethylamine lauryl sulfate, triethylamine laureth sulfate, triethanolamine lauryl sulfate, triethanolamine laureth sulfate, monoethanolamine lauryl sulfate, monoethanolamine laureth sulfate, diethanolamine lauryl sulfate, diethanolamine laureth sulfate, lauric monoglyceride sodium sulfate, sodium lauryl sulfate, potassium lauryl sulfate, potassium laureth sulfate, ammonium cocoyl sulfate, ammonium lauroyl sulfate, sodium cocoyl sulfate, sodium lauroyl sulfate, potassium cocoyl sulfate, monoethanolamine cocoyl sulfate, sodium tridecyl benzene sulfonate, sodium dodecyl benzene sulfonate, sodium C14-16 olefin sulfonate, sodium lauryl sarcosinate, sodium lauroyl sarcosinate, stearoyl sarcosine, lauryl sarcosine, cocoyl sarcosine, sodium methyl cocoyl taurate, sodium methyl lauroyl taurate, sodium lauroyl glutamate, disodium cocoyl glutamate, potassium myristoyl glutamate, TEA-cocoyl glutamate, sodium cocoyl glycinate, potassium cocoyl glycinate, sodium cocoyl alaniate, TEA-cocoyl alaninate, potassium cetyl phosphate, and mixtures thereof.

The amount of the (g) anionic surfactant in the composition according to the present invention may be 0.01% by weight or more, preferably 0.05% by weight or more, and more preferably 0.08% by weight or more, relative to the total weight of the composition.

The amount of the (g) anionic surfactant in the composition according to the present invention may be 1% by weight or less, preferably 0.5% by weight or less and more preferably 0.2% by weight or less, relative to the total weight of the composition. The amount of the (g) anionic surfactant in the composition according to the present invention may be from 0.01% to 1% by weight, preferably from 0.05% to 0.5% by weight, and more preferably from 0.08% to 0.2% by weight, relative to the total weight of the composition.

(Other Ingredients)

The composition according to the present invention may also include at least one optional or additional ingredient.

The amount of the optional or additional ingredient(s) is not limited, but may be from 0.01% to 30% by weight, preferably from 0.1% to 20% by weight, and more preferably from 1% to 10% by weight, relative to the total weight of the composition according to the present invention.

The optional or additional ingredient(s) may be selected from the group consisting of anionic, cationic, nonionic, or amphoteric polymers; cationic or amphoteric surfactants; organic or inorganic UV filters; peptides and derivatives thereof; protein hydrolyzates; swelling agents and penetrating agents; agents for combating hair loss; anti-dandruff agents; natural or synthetic thickeners; suspending agents; sequestering agents; opacifying agents; dyes; sunscreen agents; vitamins or provitamins; fragrances; preservatives, co-preservatives, stabilizers; and mixtures thereof

The composition according to the present invention may comprise one or several solid fatty substances for providing a more stabilized O/W emulsion. The solid fatty substance means a fatty compound or material which is in the form of a solid at room temperature (25° C.) under atmospheric pressure (760 mmHg). The solid fatty substance may be a fatty alcohol, preferably a linear and saturated higher alcohol, in particular a linear and saturated C₁₄-C₂₂ fatty alcohol, such as myristyl alcohol, cetyl alcohol, stearyl alcohol, arachidyl alcohol, and behenyl alcohol.

The composition according to the present invention may include one or several cosmetically acceptable organic solvents, which may be alcohols: in particular monovalent alcohols such as ethyl alcohol, isopropyl alcohol, benzyl alcohol, and phenylethyl alcohol; diols such as ethylene glycol, propylene glycol, and butylene glycol; other polyols such as glycerol, sugar, and sugar alcohols; and ethers such as ethylene glycol monomethyl, monoethyl, and monobutyl ethers, propylene glycol monomethyl, monoethyl, and monobutyl ethers, and butylene glycol monomethyl, monoethyl, and monobutyl ethers.

The organic solvent(s) may then be present in a concentration of from 0.01% to 20% by weight, preferably from 0.1% to 15% by weight, and more preferably from 1% to 10% by weight, relative to the total weight of the composition.

The pH of the composition according to the present invention may be controlled. The pH may be, for example, from 3 to 11, preferably from 4 to 9, and more preferably from 5 to 7. The pH may be adjusted to the desired value using at least one acidifying agent and/or at least one basifying agent.

The acidifying agents can be, for example, mineral or organic acids, for instance hydrochloric acid, orthophosphoric acid, carboxylic acid, for instance tartaric acid, citric acid, lactic acid, or sulphonic acids.

The basifying agent can be, for example, ammonium hydroxide, alkali metal hydroxide, alkali earth metal hydroxide, alkali metal carbonates, alkanolamines such as mono-, di-, and triethanolamines, and also their derivatives, preferably sodium or potassium hydroxide and compounds of the formula below:

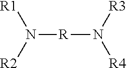

wherein

R denotes an alkylene such as propylene optionally substituted by a hydroxyl or a C₁-C₄ alkyl radical, and R₁, R₂, R₃, and R₄ independently denote a hydrogen atom, an alkyl radical, or a C₁-C₄ hydroxyalkyl radical, which may be exemplified by 1,3-propanediamine and derivatives thereof Arginine, urea, and monoethanolamine may be preferable.

The acidifying or basifying agent may be present in an amount ranging from less than 5% by weight, preferably from 1% by weight or less, and more preferably from 0.1% by weight or less, relative to the total weight of the composition.

(Form)

It is preferable that the composition according to the present invention be of the O/W type, more preferably in the form of an O/W emulsion, and even more preferably an O/W gel emulsion.

The O/W architecture or structure, which consists of oil phases dispersed in an aqueous phase, has an external aqueous phase, and therefore products based on the O/W architecture or structure are more pleasant to use because of the feeling of immediate freshness that they can provide.

If the composition according to the present invention is of the O/W type, preferably in the form of an O/W emulsion, and more preferably an O/W gel emulsion, it comprises oil phases dispersed in a continuous aqueous phase. The dispersed oil phases can be recognized as oil droplets in the aqueous phase.

It may be preferable that the composition according to the present invention of the O/W type, more preferably in the form of an O/W emulsion, and even more preferably an O/W gel emulsion, be in the form of an O/W fine emulsion, more preferably a nano- or micro-emulsion, and even more preferably a nano-emulsion.

[Preparation]

The composition according to the present invention can be prepared by mixing the ingredients (a) to (f) as well as optional ingredient (g) and/or additional optional ingredient(s) as explained above.

For example, the composition according to the present invention can be prepared by the process comprising:

(i) mixing
(f) water;
(a) at least one microcrystalline cellulose;
(d) at least one polysaccharide hydrophilic gelling agent other than the ingredient (a);
to obtain a first mixture,

(ii) optionally mixing the first mixture thus obtained in step (i) with
(g) at least one anionic surfactant,
to obtain a second mixture, and

(iii) mixing the first mixture thus obtained in step (i) or the second mixture thus obtained in step (ii) with
(b) at least one nonionic surfactant of ester type;
(c) at least one surfactant of alkylpolyglycoside type; and
(e) at least one oil,
to prepare the composition.

The mixing step can be performed by any conventional means.

[Use and Process]

The composition according to the present invention may preferably be used as a cosmetic composition, more preferably a skin cosmetic composition, and even more preferably a skincare cosmetic composition.

The composition according to the present invention is stable for a long period of time even under low and high temperatures.

The composition according to the present invention is sprayable to dispense or discharge a fine mist of the composition according to the present invention.

The composition according to the present invention can provide a fresh sensation after application, in particular a fresh sensation immediately after application. Therefore, the fresh sensation can start from the beginning of use and continue during use.

The composition according to the present invention can provides no sticky feeling after application, and therefore, can provide a smooth skin finish.

The skin here encompasses face skin, neck skin, and the scalp. The composition according to the present invention may also be used for mucosae such as the lips, and the like.

The composition according to the present invention can be used as it is (as a topical product), or can be used by being impregnated into a porous substrate such as a non-woven fabric preferably made from cellulose fibers to prepare a cosmetic product such as a cosmetic mask.

In particular, the composition according to the present invention may be intended for application onto a keratin substance such as the skin or lips, preferably the skin. Thus, the composition according to the present invention can be used for a cosmetic process for the skin or lips, preferably the skin.

It is preferable that the composition according to the present invention be used for skincare, not for skin makeup. In other words, it is preferable that the composition according to the present invention be used for skincare products such as a lotion (in particular, a milky lotion) and a cream, not for skin makeup products such as a foundation.

The cosmetic process for a keratin substance such as the skin, according to the present invention may comprise, at least, the step of applying onto the keratin substance the composition according to the present invention.

It is preferable that the application of the composition according to the present invention be performed by spraying the composition according to the present invention.

Thus, it is preferable that the composition according to the present invention be in a sprayable product. For example, the composition according to the present invention may be in a container, such as a bottle, a tube, and a vessel, which is equipped with a mist-generating means including, for example, a nozzle and a mechanical pump.

The composition according to the present invention may be in a so-called aerosol product. For example, the composition according to the present invention may be in a container, such as a bottle, a tube, and a vessel, which is equipped with a mist-generating means including, for example, a nozzle and a propellant such as volatile hydrocarbons and dimethylether. However, in view of environmental factors, it is preferable that the composition according to the present invention be used in non-aerosol products.

According to another aspect, the present invention also relates to the use of

(a) at least one microcrystalline cellulose;

(b) at least one nonionic surfactant of ester type;

(c) at least one surfactant of alkylpolyglycoside type; and

(d) at least one polysaccharide hydrophilic gelling agent other than the ingredient (a), in a composition comprising (e) oil and (f) water, in order to make the composition have good sprayability and stability, and being capable of providing a smooth skin finish.

### EXAMPLES

The present invention will be described in a more detailed manner by way of examples. However, these examples should not be construed as limiting the scope of the present invention. The examples below are presented as non-limiting illustrations in the field of the present invention.

### Examples 1-2 and Comparative Examples 1-4

The following compositions according to Examples 1-2 and Comparative Examples 1-4 shown in Table 1, were prepared by mixing the ingredients shown in Table 1 as follows. The numerical values for the amounts of the ingredients shown in Table 1 are all based on “% by weight” of the ingredients shown in the table.

TABLE 1

| | | | | Comp. | Comp. | Comp. | Comp. |
|---|---|---|---|---|---|---|---|
| Ingredients | | Ex. 1 | Ex. 2 | Ex. 1 | Ex. 2 | Ex. 3 | Ex. 4 |
| Sodium Citrate | | 0.025 | 0.025 | 0.025 | 0.025 | 0.025 | 0.025 |
| Hydroxyacetophenone | | 0.2 | 0.2 | 0.2 | 0.2 | 0.2 | 0.2 |
| Citric Acid | | 0.01 | 0.01 | 0.01 | 0.01 | 0.01 | 0.01 |
| Phenoxyethanol | | 0.5 | 0.5 | 0.5 | 0.5 | 0.5 | 0.5 |
| Chlorphenesin | | 0.2 | 0.2 | 0.2 | 0.2 | 0.2 | 0.2 |
| Water | | qsp 100 | qsp 100 | qsp 100 | qsp 100 | qsp 100 | qsp 100 |
| Glvcerin | | 20 | 20 | 20 | 20 | 20 | 20 |
| Xanthan Gum (and) Ceratonia Siliqua (Carob)Gum *¹ | | 0.01 | 0.01 | 0.01 | 0.01 | 0.01 | — |
| Microcrystalline Cellulose (and) Cellulose Gum *² | | 1.2 | — | — | 1.2 | 1.2 | 1.2 |
| Microcrystalline Cellulose (2 wt % aqueous dispersion) *³ | | — | 10 | — | — | — | — |
| Potassium Cetyl Phosphate | | 0.1 | 0.1 | 0.1 | 0.1 | 0.1 | 0.1 |
| Caprylic/Capric Triglyceride | | 1.5 | 1.5 | 1.5 | 1.5 | 1.5 | 1.5 |
| Dimethicone | | 2.5 | 2.5 | 2.5 | 2.5 | 2.5 | 2.5 |
| Cetearyl Alcohol (and) Cetearyl Glucoside *⁴ | | 0.7 | 0.7 | 0.7 | 0.7 | — | 0.7 |
| Polyglyceryl-6 Distearate (and) Jojoba Esters (and) Cetyl Alcohol | | 1.5 | 1.5 | 1.5 | — | 1.5 | 1.5 |
| (and) Polyglyceryl-3 Beeswax *⁵ | | | | | | | |
| Tocopherol | | 0.1 | 0.1 | 0.1 | 0.1 | 0.1 | 0.1 |
| Sprayability | State of Spray | Excellent | Excellent | Excellent | Excellent | Good | Excellent |
| | Refreshing Feeling Immediately after being | Excellent | Excellent | Excellent | Good | Good | Excellent |
| | Sprayed Out | | | | | | |

| | | | | | | |
|---|---|---|---|---|---|---|
| Non-Sticky Feeling after Application (Smooth Skin Finish) | Excellent | Excellent | Excellent | Poor | Good | Good |
| Temperature Stability (4-45° C., 2 months) | Excellent | Excellent | Very | Very | Very | Very |
| | | | Poor | Poor | Poor | Poor |

*¹ NOMCORT ® CG (The Nisshin Oillio Group, Ltd.): XANTHAN GUM/CERATONIA SILIQUA (CAROB) GUM = 50/50 (parts by weight)
*² Avicel ® PC 611 (FMC Corporation): MICROCRYSTALLINE CELLULOSE/CELLULOSE GUM = 85/15 (parts by weight)
*³ RHEOCRYSTA ® C-2SP (DKS Co. Ltd.): MICROCRYSTALLINE CELLULOSE/WATER/PHENOXYETHANOL = 2/97/1 (parts by weight)
*⁴ Montanov ™ 68 (SEPPIC): CETEARYL ALCOHOL/CETEARYL GLUCOSIDE = 80/20 (parts by weight)
*⁵ Emulium ® Mellifera (GATTEFOSSE): POLYGLYCERYL-6 DISTEARATE/JOJOBA ESTERS/CETYL ALCOHOL/POLYGLYCERYL-3 BEESWAX = 64/19/8.5/8.5 (parts by weight)

[Evaluations]

{Sensory Evaluation}

5 professional panelists evaluated “sprayability” and “no sticky feeling after application (smooth skin finish)” of the compositions according to Examples 1-2 and Comparative Examples 1-4. The compositions according to Examples 1-2 and Comparative Examples 1-4 were evaluated as follows.

(Sprayability)

(1) State of Spray

Each panelist sprayed out each of the compositions according to Examples 1-2 and

Comparative Examples 1-4 filled in a commercially-available spray container*, and graded from 1 (poor) to 5 (excellent), which was then classified in the following 4 categories based on the average of the grade.
Excellent (5.0 to 4.0): The spraying composition can be sprayed from the nozzle and form an excellent fine mist.
Good (3.9 to 3.0): The spraying composition can be sprayed from the nozzle and form a fine mist.
Poor (2.9 to 2.0): Although the spraying composition can be sprayed from the nozzle, it cannot form a fine mist.
Very Poor (1.9 to 1.0): The spraying composition cannot be sprayed from the nozzle, that is, the spraying composition cannot form a mist.

The results are shown in Table 1.
Commercially-available spray container: Sprayer: SP-22 (ALBEA) with bottle

(2) Refreshing Feeling Immediately after being Sprayed Out

Each panelist sprayed out each of the compositions according to Examples 1-2 and Comparative Examples 1-4 filled in a commercially-available spray container* onto their faces to evaluate the refreshing feeling immediately after being sprayed out for each composition, and were graded from 1 (poor) to 5 (excellent), which was then classified in the following 4 categories based on the average of the grade.
Excellent (5.0 to 4.0): Very refreshing feeling is felt after being sprayed out.
Good (3.9 to 3.0): Refreshing feeling is felt after being sprayed out.
Poor (2.9 to 2.0): Little refreshing feeling is felt after being sprayed out.
Very Poor (1.9 to 1.0): No refreshing feeling is felt after being sprayed out.

The results are shown in Table 1.
Commercially-available spray container: Sprayer: SP-22 (ALBEA) with bottle

(Non-Sticky Feeling after Application (Smooth Skin Finish))

Each panelist sprayed out each of the compositions according to Examples 1-2 and Comparative Examples 1-4 filled in a commercially-available spray container* onto their faces to evaluate the no sticky feeling after application for each composition, and graded from 1 (poor) to 5 (excellent), which was then classified in the following 4 categories based on the average of the grade.
Excellent (5.0 to 4.0): Very smooth feeling is felt after application.
Good (3.9 to 3.0): Smooth feeling is felt after application.
Poor (2.9 to 2.0): Little smooth feeling is felt after application.
Very Poor (1.9 to 1.0): Sticky feeling is felt after application.

The results are shown in Table 1.
Commercially-available spray container: Sprayer: SP-22 (ALBEA) with bottle

{Temperature Stability}

Each of the compositions according to Examples 1-2 and Comparative Examples 1-4 was filled into four glass bottles, and each of the glass bottles was held under temperature conditions of 4° C., 25° C., 40° C., and 45° C., respectively, for 2 months.

Each glass bottle was then investigated for the degree of change (in terms of transparency, color, odor, and pH), and evaluated by the following criteria.
Excellent: Almost the same condition as production.
Good: Change in color, odor, and pH could be somewhat observed. No separation aspect was observed.
Poor: Change in color, odor, and pH could be clearly observed. Separation aspect could also be clearly observed.
Very Poor: Change in color, odor, and pH could be remarkably noticed. Separation aspect could be remarkably noticed.

The results are shown in Table 1.

In view of Examples 1 and 2 with respect to Comparative Example 1, it can be understood that the (a) microcrystalline cellulose can enhance the stability of the composition according to the present invention under a variety of temperature conditions.

In view of Examples 1 and 2 with respect to Comparative Example 2, it can be understood that the (b) nonionic surfactant of ester type can also enhance the stability of the composition according to the present invention, and render the composition according to the present invention capable of providing a refreshing feeling immediately after being sprayed out and a non-sticky feeling after application or smooth skin finish, in particular non-sticky feeling after application.

In view of Examples 1 and 2 with respect to Comparative Example 3, it can be understood that the (c) surfactant of alkylpolyglycoside type can also enhance the stability of the composition according to the present invention, and render the composition according to the present invention capable of spraying a fine mist and providing a refreshing feeling immediately after being sprayed out and a non-sticky feeling after application or smooth skin finish.

In view of Examples 1 and 2 with respect to Comparative Example 4, it can be understood that the (d) polysaccharide hydrophilic gelling agent other than the ingredient (a) can also enhance the stability of the composition according to the present invention, and render the composition according to the present invention capable of providing a refreshing feeling immediately after being sprayed out.

Accordingly, it can be understood from Table 1 that ingredients (a) to (d) are necessary to realize a composition, in particular in the form of an O/W emulsion, which has good sprayability and stability, and can provide a smooth skin finish.

1. A composition, preferably in the form of an O/W emulsion, comprising:
(a) at least one microcrystalline cellulose;
(b) at least one nonionic surfactant of ester type;
(c) at least one surfactant of alkylpolyglycoside type;
(d) at least one polysaccharide hydrophilic gelling agent other than the ingredient (a);
(e) at least one oil; and
(f) water.

2. The composition according to claim 1, wherein the (a) microcrystalline cellulose is in the form of a particle.

3. The composition according to claim 1, wherein the amount of the (a) microcrystalline cellulose in the composition is from 0.05% to 5% by weight, relative to the total weight of the composition.

4. The composition according to claim 1, wherein the (b) nonionic surfactant of ester type is selected from the group consisting of monounsaturated esters, polyglyceryl diesters, and mixtures thereof.

5. The composition according to claim 1, wherein the (b) nonionic surfactant of ester type is a mixture comprising:
i) at least one monounsaturated ester of formula (A)
R¹—C(O)—O—R² (A)
wherein
R¹ and R² represent, respectively, a C₁₈ to C₄₄ fatty chain, at least one of R¹ or R² is monounsaturated;
ii) at least one polyglyceryl diester of formula (B)
R³—C(O)—(O—CH₂—CH(OH)—CH₂)ₙ—O—C(O)—R⁴ (B)
wherein
R³ and R⁴ represent, respectively, a saturated C₁₈ to C₄₄ fatty chain, linear or branched, and n is an integer between 2 and 6; and
iii) at least one C₁₀-C₃₀ fatty alcohol, preferably, the (b) nonionic surfactant of ester type is a mixture comprising at least one monounsaturated ester of formula (A) wherein R¹ and R² represent, respectively, a C₁₈-C₃₀ fatty chain, and at least one of R₁ or R₂ is monounsaturated; at least one polyglyceryl diester of formula (B) wherein, R³ and R⁴ each represent a saturated C₂₀ to C₃₄ fatty chain, linear or branched; and cetyl alcohol.

6. The composition according to claim 1, wherein the (b) nonionic surfactant of ester type further comprises a diester of a C₁₄-C₂₂ fatty acid with a polyglycerol and/or a fatty alcohol containing from 10 to 30 carbon atoms.

7. The composition according to claim 1, wherein the (b) nonionic surfactant of ester type is a mixture of polyglyceryl-6 distearate, jojoba esters, polyglyceryl-3 beeswax, and cetyl alcohol.

8. The composition according to claim 1, wherein the amount of the (b) nonionic surfactant of ester type in the composition is from 0.05% to 10% by weight, relative to the total weight of the composition.

9. The composition according to claim 1, wherein the (c) surfactant of alkylpolyglycoside type is selected from the compound of formula (I):
R(O)(G)ₓ (I)
in which the radical R is a linear or branched C₁₂-C₂₂ alkyl radical, x ranges from 1 to 5 and G is a saccharide residue chosen from the group of glucose, dextrose, saccharose, fructose, galactose, maltose, maltotriose, lactose, cellobiose, mannose, ribose, dextran, talose, allose, xylose, levoglucan, cellulose and starch; preferably, G is glucose.

10. The composition according to claim 1, wherein the amount of the (c) surfactant of alkylpolyglycoside type in the composition is from 0.01% to 5% by weight, relative to the total weight of the composition.

11. The composition according to claim 1, wherein the (d) polysaccharide hydrophilic gelling agent is selected from xanthan gum, carob gum and mixtures thereof.

12. The composition according to claim 1, wherein the amount of the (d) polysaccharide hydrophilic gelling agent in the composition is from 0.0005% to 0.5% by weight, relative to the total weight of the composition.

13. The composition according to claim 1, wherein the amount of the (e) oil in the composition is from 0.1% to 20% by weight, relative to the total weight of the composition.

14. The composition according to claim 1, wherein the amount of the (f) water in the composition is from 50% to 95% by weight, relative to the total weight of the composition.

15. The composition according to claim 1, wherein the composition further comprises (g) at least one anionic surfactant.

16. The composition according to claim 15, wherein the amount of the (g) anionic surfactant(s) in the composition is from 0.01% to 1% by weight, relative to the total weight of the composition.

17. The composition according to claim 1 wherein the composition is a cosmetic composition.

18. A cosmetic process for a keratin substance, prcfcrably skin, comprising applying to the keratin substance the composition according to claim 1.
